(19) United States
(12) Patent Application Publication
(10) Pub. No.: US 2015/0343159 A1
(43) Pub. Date: Dec. 3, 2015

(54) Inhaler Device

(71) Applicant: Glaxo Group Limited, Brentford (GB)

(72) Inventors: Philip William FARR, Ware (GB); John DEAMER, Barnston (GB); Mark Gregory PALMER, Ware (GB); Alan Anthony WILSON, Ware (GB); Stephen Maurice Pitson, Sturmer (GB)

(21) Appl. No.: 14/410,232

(22) PCT Filed: Jul. 4, 2013
(86) PCT No.: PCT/EP2013/064133

§ 371 Date: Dec. 22, 2014

Related U.S. Application Data
(60) Provisional application No. 61/668,112, filed on Jul. 5, 2012.

(51) Int. Cl. A61M 15/00

(52) U.S. Cl. CPC A61M 15/0035; A61M 15/004; A61M 15/0045; A61M 15/0026; A61M 2202/064

(57) ABSTRACT

There is provided an inhaler device comprising a housing, a mouthpiece, a seat for receiving a unit dose blister and a punch for piercing a lid of the unit dose blister. The housing comprises a base and a lid pivotally joined by a hinge, such that the lid is pivotable from a first ‘closed’ position in which it abuts the base to define a cavity, to a second ‘open’ position in which the cavity can be accessed. The seat and the punch are adapted to lie within the cavity when the lid is in the first ‘closed’ position and moving the lid from the ‘open’ position to the ‘closed’ position causes the punch to pierce a lid of a unit dose blister received in the seat. Only the lid of the unit dose blister is pierced.

### FIELD OF THE INVENTION

The present invention relates to an inhaler device suitable for the delivery of medication and a punch suitable for use within such an inhaler device. It is particularly concerned with a dry powder inhaler device for delivery of dry powder medication from a unit dose blister to the lungs of a patient. The dry powder medication may carry a topical medication, such as salbutamol for the treatment of asthma, or systemic medications, such as inhalable insulin for the treatment of diabetes, or vaccines, or inhalable oxytocin for the treatment of postpartum haemorrhage.

### BACKGROUND OF THE INVENTION

A number of different inhaler types are known. A first type, called a reservoir inhaler, stores multiple doses of dry powder medication in bulk. The inhaler is provided with a metering device, often in the form of a metering drum, which meters a dose of the medication from the bulk store for inhalation by a user of the device.

A further type of inhaler stores dry powdered medication in the form of pre-metered, discrete, doses. Typically, the device houses a blister pack comprising multiple blisters, with each blister holding a unit dose of the medication. The blister pack is conveniently arranged as an elongate strip, or disk, which is advanced, and opened, by a mechanism within the inhaler prior to inhalation of the medication. Opening is typically achieved by peeling or puncturing the blister pocket id to access the medication contained therein, or by rupturing the pocket and spilling the medication into a receiving chamber. U.S. Pat. No. 5,873,360 describes a device in which a blister strip has a lid which is peeled apart from a pocket of the blister strip to allow air to flow into the pocket, in order to aerosolize medication held within the pocket.

Both reservoir type and multiple discrete dose type inhaler devices are relatively complex devices and consequently have a cost of goods that is economic only when the device is loaded with a large number of doses of medication. For example the DISKUS device, manufactured by GLAXOSMITHKLINE, provides a month's supply of twice-daily medication, by providing 60 pre-metered doses of medication in a blister strip held within the device.

For a number of reasons, it is sometimes desirable to provide smaller numbers of discrete doses of an inhaled medication than is economically possible with the inhaler types described above. For example, the stability of the medication may preclude long term storage under normal conditions. Another reason may be that the patient cannot afford to purchase long term supplies of the medication, and so prefers to purchase smaller volumes of doses of the medication, as circumstances permit. In fact, patients may only be able to afford to buy a single dose of the inhaled medication at a time.

Unit dose inhaler devices are known in the art, such as the ROTAHALER device manufactured by Allen and Hanburys Limited. Such a device, described in U.S. Pat. No. 4,353,365, typically uses a two-part capsule for the delivery of a pre-metered unit dose of medication which is inserted into the device by a patient. The capsule is then separated, by patient operation of the device, to distribute powder within a chamber of the device for delivery to the lung of the patient when the patient subsequently inhales through the device.

Such unit dose inhaler devices typically comprise a number of separate parts to enable disassembly for insertion of the capsule, and for the separation operation. They further include a separately formed perforated guard which prevents inhalation of fragments of the broken capsule. The capsules of such devices are prone to ingress of moisture, and typically require a secondary package to provide a moisture barrier, increasing the cost of goods.

European Patent 0 129 985 discloses a unit dose inhaler in which a drug is released from a unit dose blister by driving a single spike through both the lid and the base of the blister. Such a method of release requires the application of a large force to the device during release in order to drive the spike through the thickness of the material of the blister, particularly the blister base which must be sufficiently robust to hold a pocket shape. Furthermore, it is difficult to remove the blister from the spike due to plastic deformation of the blister about the spike.

The forces required to pierce the blister, and remove the spike from the blister lead to a much more robust device than would otherwise be required, resulting in greater cost, weight and bulk.

It is an objective of the present invention to provide an inhaler device suitable for the delivery of medication from a unit dose blister which has improved economics compared to inhalers of the prior art. Preferably the inhaler device has a reduced parts count, and is manufactured from three parts or less.

It is a further objective of the present invention to provide a punch for an inhaler device which is suitable for piercing the lid foil of a unit blister dose which is optimized to provide improved efficiency for delivery of an inhalable, aerosolizable, medication held within the blister dose.

It will be understood that the term unit dose is intended in the present context to describe a pre-metered dose of medication which comprise all or a suitable fraction of a quantity recommended to be taken at a particular time by a patient. In other words, an effective quantity of a medication could be delivered by more the inhalation of plural unit doses.

### SUMMARY OF THE INVENTION

According to one aspect of the invention, there is provided an inhaler device comprising;
a housing,
a mouthpiece,
a seat for receiving a unit dose blister, the unit dose blister comprising a blister pocket and a blister lid,
and a punch for piercing the lid of a unit dose blister when received in the seat,

wherein the housing comprises a base and a lid pivotally joined by a hinge, such that the housing lid is pivotable from a first ‘closed’ position in which it abuts the housing base to define a cavity, to a second ‘open’ position in which the cavity can be accessed,

wherein the seat and the punch are adapted to lie within the cavity when the lid is in the first ‘closed’ position and

wherein moving the housing lid from the ‘open’ position to the ‘closed’ position causes the punch to pierce the lid of the unit dose blister when received in the seat

and wherein only the lid of the unit dose blister is pierced.

Suitably the punch projects from a first side of the housing into the cavity when the lid is in the closed position.

Suitably, the seat projects from an opposing side of the housing into the cavity when the lid is in the closed position.

Suitably, the seat projects from the housing base and the punch projects from the housing lid.

In one aspect, the punch cooperate with the unit dose blister lid and unit dose blister pocket to create a filter which selectively retains medication particles of a predetermined size during use of the inhaler device.

Suitably, the filter is formed by an unpierced annular region of the unit dose blister lid.

Suitably, the annular region comprises about 65% of the area of a puncturable disc region of the unit dose blister lid. The puncturable disc region comprises the blister lid excluding an annular collar, the annular collar providing a location feature for insertion of the unit dose blister lid into the device.

In one aspect, the punch comprises a first piercing blade and a second piercing blade, and the first and second piercing blades are arranged such that movement of the housing lid from the second position to the first position causes the first piercing blade to engage and pierce the lid of the unit dose blister before the second piercing blade engages and pierces the lid.

Preferably, the first piercing blade is an upstream blade, configured to pierce an inlet aperture in the blister lid, and the second piercing blade is a downstream blade, configured to pierce an exit aperture in the blister lid.

In one aspect, the at least one piercing blade comprise a semi-oval planar element.

In one aspect, the first piercing blade and second piercing blades diverge from one another.

In one aspect, the first piercing blade and second piercing blade share a common linear base and are angled apart such that the first and second piercing blade form an inverted ‘V’ when viewed along the linear base

In one aspect, at least one piercing blade cooperates with the unit dose blister lid and the unit dose blister pocket to define a channel through which an airflow can enter and/or exit the unit dose blister.

Suitably, the piercing blade contacts the unit dose blister pocket to divide the channel.

In a further aspect, the first piercing blade cooperates with the lid of the unit dose blister and the unit dose blister pocket to define a first channel and the second piercing blade cooperates with the lid of the unit dose blister and the wall of the unit dose blister to define a second channel, and wherein air enters the unit dose blister via the first channel and exits the unit dose blister via the second channel.

Suitably, the first piercing blade contacts the unit dose blister to divide the first channel, and the second piercing blade contacts the wall of the unit dose blister to divide the second channel, and air enters the unit dose via the divided first channel, and exits the unit dose blister via the divided second channel.

In one aspect, the housing cooperates with the lid of the unit dose blister to form a dosing channel which divides an airflow through the device into a pocket airflow, and a bypass airflow, wherein the pocket airflow aerosolizes a powder held within the unit dose blister and the bypass airflow circumvents the unit dose blister.

The housing, the mouthpiece, the seat, and the punch may be formed as a single component, which is to say that the complete device, absent the mouthpiece cover, may be formed as a single component.

Suitably, the housing, the mouthpiece, the seat and the punch are formed by a single-shot or multi-shot injection moulding process.

In one aspect, the inhaler device further comprises a mouthpiece cover which can be attached to the inhaler device to enclose the mouthpiece, wherein the housing lid can be moved from the ‘open’ position to the ‘closed’ position without removing the mouthpiece cover such that the device cavity is substantially sealed from the external environment when the unit dose blister is fully opened by the punch.

Suitably, the mouthpiece is attached to the housing by a lanyard.

In one aspect, an inlet provided to the housing is covered by the mouthpiece when it is attached to the housing to enclose the mouthpiece.

In one aspect, the mouthpiece comprises a duct having a proximal end in flow communication with the housing, and a distal free end, wherein the housing communicates with the duct via an aperture which is smaller than the duct to minimise contact of powder laden air with an inner wall of the duct.

Suitably, the mouthpiece depends from a first region of a curved mouthpiece bulkhead, and an air inlet is provided to a second region of the curved mouthpiece bulkhead, wherein the air inlet is set back from the first region of the bulkhead towards the inhaler cavity.

Suitably the mouthpiece comprises a duct having a proximal end in flow communication with the housing, and a distal free end, wherein the housing communicates with the duct via an aperture which is smaller than the duct to minimise contact of powder laden air with an inner wall of the duct.

Suitably, the device further comprises a unit dose blister retainer which holds the blister in a predetermined relationship with the housing base as the punch is withdrawn from the blister by moving the housing lid from the ‘closed’ position to the ‘open’ position.

Suitably, the retainer comprises a hook, which may be formed integral with the device housing, preferably the device housing base.

Alternatively, the unit dose blister retainer may comprise a plate, provided to the housing lid, which plate is mounted to, and sprung apart from, the housing lid so as to urge the blister pocket away from the punch.

According to a further aspect of the present invention, there is provided a punch for an inhaler device, the punch adapted to pierce a blister comprising a base sheet defining a pocket, a pocket wall, and a lid covering the pocket,

wherein the punch comprises a downstream piercing blade adapted to pierce and define an exit aperture in the lid, wherein the exit aperture is spaced apart from the pocket wall to define an overhang region of the lid,

and wherein the downstream blade is further adapted to enter the pocket of the blister after piercing the lid to define a nozzle in cooperation with the wall of the blister pocket, such that when an airflow is generated through the pocket towards the exit aperture, the nozzle directs the airflow towards the overhang region of the lid so that it follows a torturous path before reaching the aperture.

Suitably, the punch further comprises an upstream piercing blade adapted to pierce and define an inlet aperture in the lid.

Suitably, the first downstream blade is wider than the upstream blade, preferably about 40% wider.

Suitably, at least one piercing blade comprises a semi-oval planar element.

Suitably, the downstream piercing blade and upstream piercing blades diverge from one another.

Suitably, the downstream piercing blade and upstream piercing blade share a common linear base and are angled apart such that the downstream and upstream piercing blade form an inverted ‘V’ when viewed along the linear base

Suitably, the downstream piercing blade is adapted to contact the wall of the blister pocket after the punch has pierced the blister lid.

Suitably, the upstream piercing blade is adapted to contact the wall of the blister pocket after the punch has pierced the blister lid.

### BRIEF DESCRIPTION OF FIGURES

FIG. 1 shows a perspective view of an inhaler device according to an aspect of the present invention in a first ‘closed’ position, with a mouthpiece cover in place.

FIG. 2 shows a perspective view of the inhaler device of FIG. 1 in a second, ‘open’ position, with the mouthpiece cover removed from a housing of the device.

FIG. 3 shows a normal view on arrow A of FIG. 2 on a mouthpiece of the device.

FIG. 4 shows a perspective view of the inhaler device of FIG. 1 with a first unit dose blister which has been pierced by the inhaler device, and a second, unpierced, unit dose blister stored within the device.

FIG. 5 shows a perspective view of the inhaler device of FIG. 1, substantially as FIG. 4, but with a third, unpierced, unit dose blister stored within the device.

FIG. 6 shows a punch according to an aspect of the present invention, as used in the device of FIG. 1, in more detail.

FIG. 7 shows a cross-section view on the section marked B-B on the punch of FIG. 6.

FIG. 8 shows a section view on the section marked C-C on the inhaler device of FIG. 1. The device is shown loaded with a unit dose blister which has been pierced.

FIG. 9 shows a perspective view of a unit dose blister after piercing by the punch of the inhaler device of FIG. 1.

FIG. 10 shows a plan view on the pierced lid of the unit dose blister of FIG. 9.

FIG. 11 shows a cross section of the pierced unit dose blister of FIG. 9.

FIG. 12 shows a cross-section view of the inhaler device of FIG. 1 on the section marked D-D (coincident with the longitudinal axis of the device). The device is shown loaded with a unit dose blister which has been pierced.

FIG. 13 shows a view from within a central region of the pierced unit dose blister loaded in the device of FIG. 12, looking in the upstream direction.

FIG. 14 shows a view from within a central region of the pierced unit dose blister loaded in the device of FIG. 12, looking in the downstream direction.

FIG. 15 shows a perspective view on the device of FIG. 8 section in the plane indicated by dashed line E-E with a schematic illustration of the airflow through the device in use. The arrows represent the internal airflow generated when a user inhales through the device.

FIG. 16 shows a close-up view on part of the cross-section shown at FIG. 8, with a schematic illustration of airflow through the device in use. The arrows represent the internal airflow generated when a user inhales through the device.

FIG. 17 shows detailed view of an alternative punch according to an aspect of the invention, for use in the inhaler device of FIG. 1.

FIG. 18 shows a view from within a central region of a pierced unit dose blister in the upstream direction

FIG. 19 shows cross section of the alternative punch shown in FIG. 17.

FIG. 20 shows a view on the outlet of a modified mouthpiece for use with the inhaler device of FIG. 1

FIG. 21 shows a view on an inhaler device provided with a modified housing lid

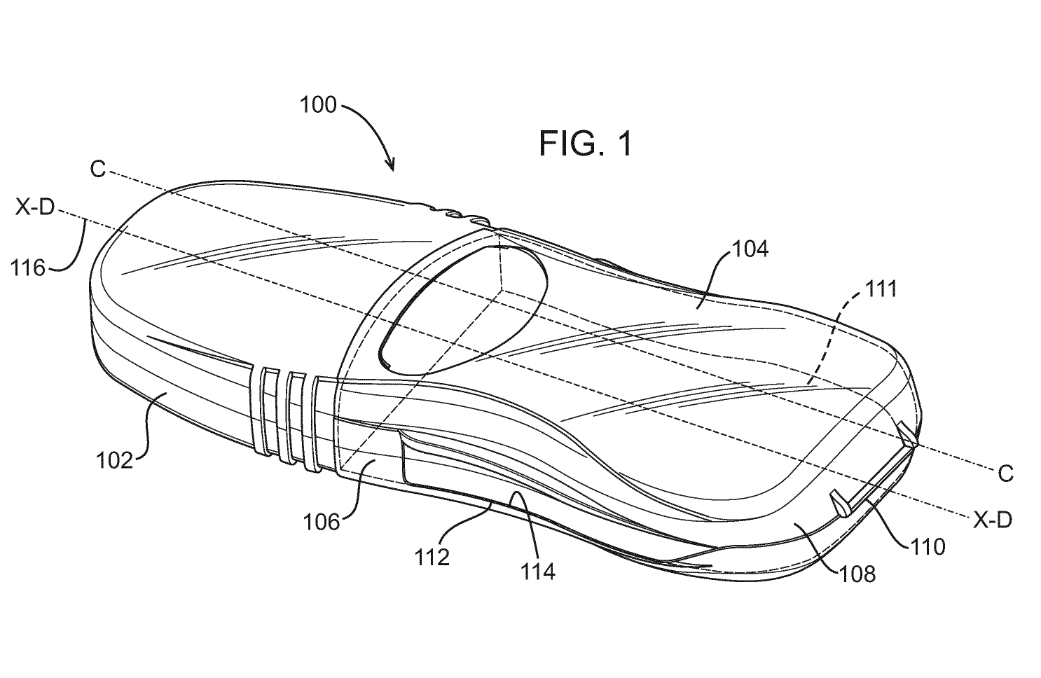

FIGS. 1-22 are based upon engineering drawings used for production of the device. Hence the drawings are to scale and representative of the geometry used in an inhaler and or punch according to the present invention.

Detailed Description of the Exemplary Embodiment of the Invention.

Turning to FIG. 1, an inhaler device 100 is shown in a closed position. Mouthpiece cover 102 is shown removably attached to a housing 104 of the device 100 in order to cover a mouthpiece (not shown) of the device 100, hence only the housing 104 and mouthpiece cover 102 are visible. The housing comprises a housing base 106 and a housing lid 108, pivotally joined to the base 106 by a hinge 110.

In the first ‘closed’ position of FIG. 1 the inhaler lid 108 lies against the inhaler base 106 to define an internal cavity 111 shown in dashed outline. Abutting surfaces 112, 114 of the base 106 and lid 108 respectively abut one another in the closed position shown.

The inhaler device 100 has a longitudinal axis 116, marked X-X.

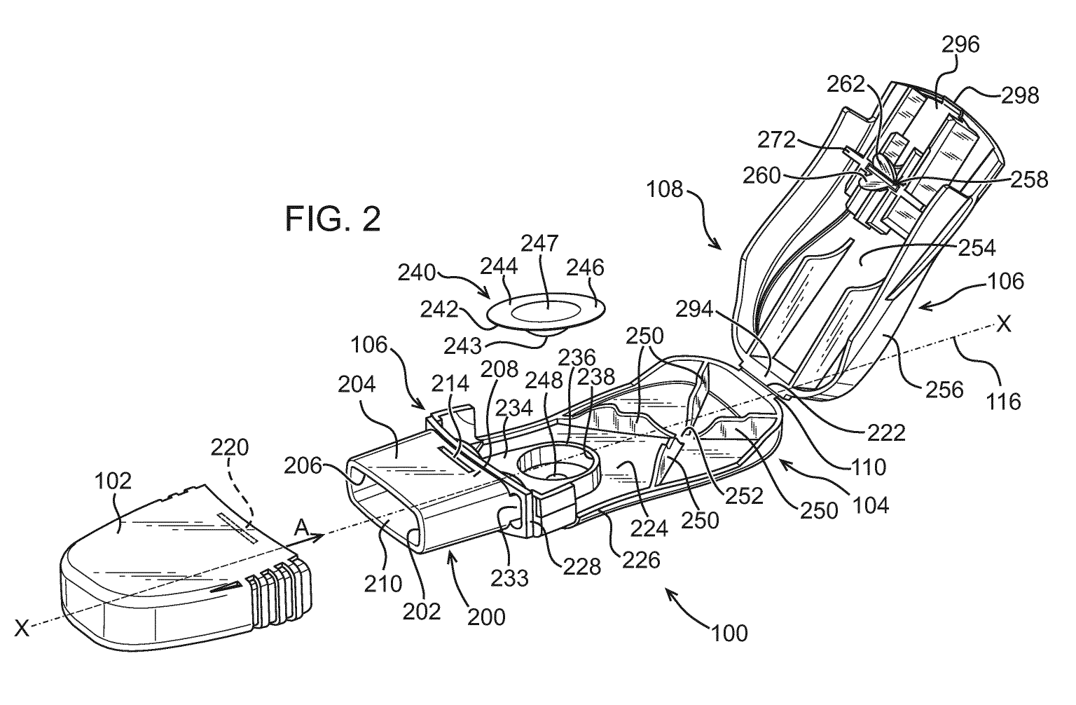

Turning to FIG. 2, the inhaler device is shown in an open position, with the mouthpiece cover 102 detached from the housing 104 so that the inhaler mouthpiece 200 is visible.

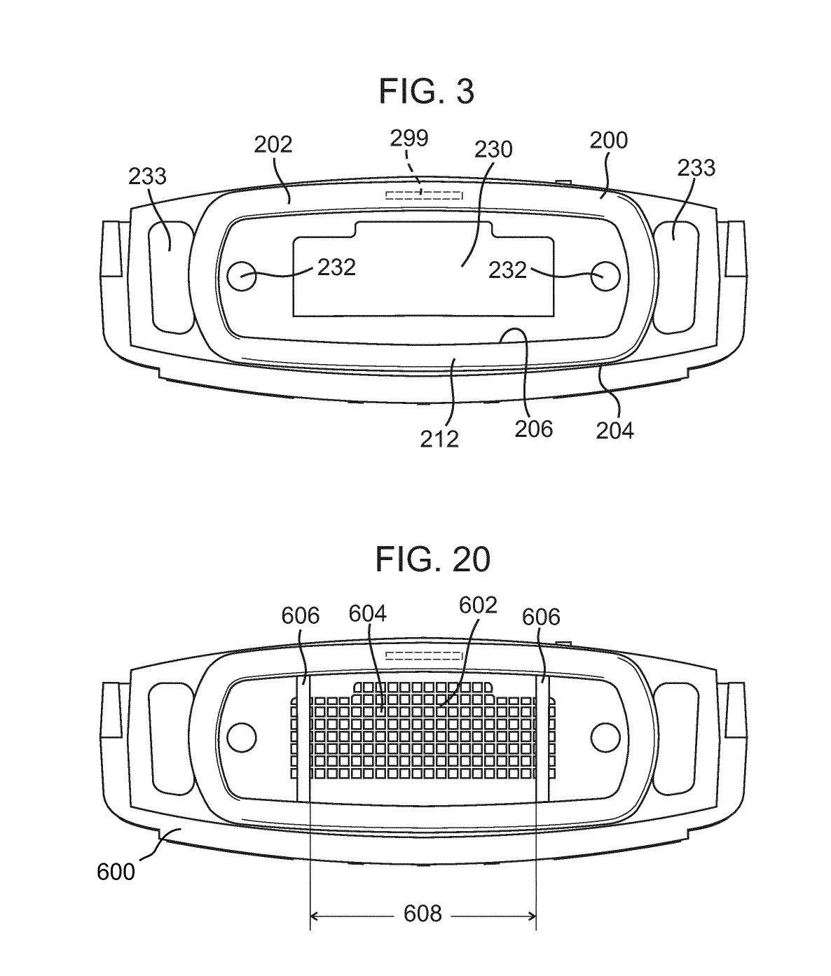

The mouthpiece 200 is an open ended duct defined by a single wall 202 which comprises an external surface 204 and an internal surface 206. The duct projects from the housing base 106 in the direction of the longitudinal axis 116, from a proximal end 208 adjoining the housing base 106 to an open distal end 210. The open end 210 of the duct has an elongate barrel-shaped end-section 212, shown more clearly at FIG. 3, such that a patient can readily seal their mouth about the mouthpiece 200 to ensure an airtight seal. The cross section 212 is maintained along the length of the mouthpiece 200, from the proximal end 208 to the open distal end 210.

Returning to FIG. 2, the mouthpiece 200 is provided at its proximal end 208 with a pair of concave grooves 214, formed in the external surface 204 of the mouthpiece 200. The grooves 214 are disposed on an upper surface of the mouthpiece and on the opposite lower surface so that only a first notch 214 is visible in FIG. 2.

The mouthpiece cover 102 is provided with pair of internal cooperating projections 220, shown in dashed outline, which engage the grooves 214 formed in the mouthpiece 200. These securely locate the mouthpiece cover 102 over the mouthpiece 200 in a snap-fit type arrangement such that the mouthpiece cover 102 is easily attached to the mouthpiece 200, but increased, deliberate, effort is required to remove the cover 102, This prevents accidental removal of the cover 102 from the device 100.

In the second ‘open’ position of FIG. 2, the cavity 111 defined by the base 106 and lid 108 can be accessed. The device 100 is moved from the closed position of FIG. 1 to the ‘open’ position of FIG. 2 by pivoting the housing lid 108 away from the housing base 106 about the hinge 110. The hinge 110 comprises a locally thinned web 222 between the housing base 104 and housing lid 106 which enables pivoting movement between the two housing parts. Such a hinge is known in the art as a living hinge. The living hinge allows the manufacture of the housing base 106 and housing lid 108 as a single unit from a plastic via injection moulding. In the present embodiment the entire device 100, absent the mouthpiece cover, is injection moulded as a single component. The device 100 is injection moulded in polypropylene, but other suitable materials may be used.

Structure—Housing Base

The housing base 106 comprises a bottom plate 224 which is bowed such that it bulges outwards away from the housing cavity 111. The bottom plate 224 is of approximately rectangular plan form, having a major axis parallel to the longitudinal axis 116 of the device 100. The base 106 has an upstanding perimeter wall 226 which extends upwards from the periphery of the bottom plate 224, and which form a continuous perimeter wall 226 of varying height. The upper surface of the wall 226 provides the abutment surface 112 which engages the abutment surface 114 of the housing lid 108 to prevent the ingress of foreign objects into the cavity 111.

At a first end of the bottom plate 224, located opposite the hinge 110, the perimeter wall 226 extends across the full width of the base 106 to provide a mouthpiece bulkhead 228. The mouthpiece 200 projects from the bulkhead 228 away from the cavity 111. The mouthpiece bulkhead 228 is curved so that the central region of the bulkhead 228 is located further away from the cavity 111 on the axis 116 than either end of the bulkhead 228.

Referring back to FIG. 3, the bulkhead 228 is provided with a slot shaped aperture 230 which enables airflow through the bulkhead 228 from the cavity 111 to the inside of the mouthpiece 200. The aperture 230 is smaller than the cross section 212 of the mouthpiece 200, and located approximately central within it, such that, in use, an airflow through the aperture 230 has reduced contact with the internal surface 206 of the mouthpiece 200 as compared with an aperture of the same size as the duct interior 206. This is intended to minimise contact between powder laden air issuing from the aperture 230 and the inner wall 206 of the duct, and thereby reduce deposition of powder on the wall. It is intended to enhance this effect by the provision of first and second bleed holes 232 through the bulkhead 228, within the duct wall 202 on either side of the aperture 230. The bleed holes 232 provide a source of clean, i.e. non-powder laden air to sheath powder laden air issuing from the slot shaped aperture 230 in use.

Two air inlets 233 are provided in the bulkhead 228. Each inlet is 233 is provided in a region outboard of the mouthpiece 200, one on either side of the mouthpiece 200. The inlets 233 are located so that they are covered by the mouthpiece cover 102 when it is releasably engaged to the housing 104. This helps to prevent the escape of medicament from the inlets during the piercing process set forth below. It also prevents the ingress of contaminants into the cavity 111.

The curvature of the bulkhead 228 ensures that in use, even if a patient is able to place their mouth against the bulkhead 228, they will only do so at a central region of the bulkhead 228, where the mouthpiece adjoins the bulkhead 228. This prevents the patient from accidentally occluding the inlets 233 which are set back i.e. located closer to the cavity 111, from the centre of the bulkhead 228 in the axial direction 116 of the device 100. This helps to ensure that an airflow generated through the device 100 in use, is not impeded by blockage at the device inlets 233.

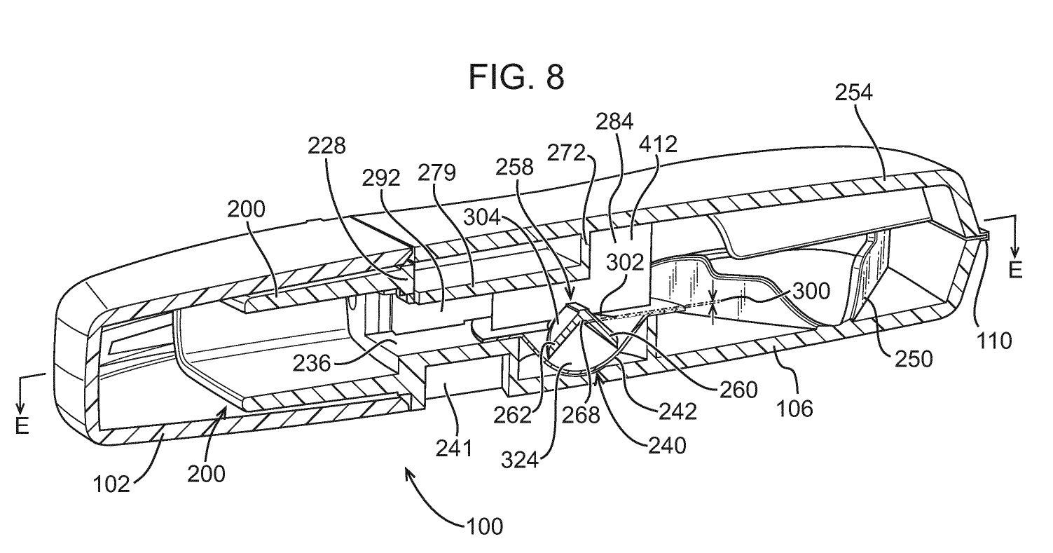

Referring back to FIG. 2, the bottom plate 224 of the housing is provided with a seat 234 which projects into the cavity 111 from the bottom plate 224. The seat 234 provides a raised platform 236 within which is formed a cylindrical recess 238 adapted to receive a unit dose blister 240. The raised platform is of the same wall thickness as the rest of the housing base 106, and a corresponding cavity 241 is formed in the exterior surface of the base 108 to enable this constant wall thickness to be maintained, as shown at FIG. 8. This ensures constant wall thickness which improves the suitability of the device 100 for injection moulding.

Structure—Unit Dose Blister

As shown at FIG. 2, the unit dose blister 240 comprises base sheet 242 comprising an aluminium-polymer laminate about 45 microns thick, in which is formed a concave blister pocket 243 having a circular perimeter. The base sheet 242 is covered by a lid 244 comprising an aluminium-polymer laminate sheet, about 25 microns thick.

The lid 244 is sealed to the base sheet 242 about the pocket 243 to provide a flat annular collar 246. The lid 244 is unsupported by the base sheet 242 inboard of the collar 246, thereby providing a thin puncturable disc 247 of lid foil over the blister pocket 243. The pocket 243 and the lid 244 together define a sealed cavity for the storage of medication (not shown).

The unit dose blister 240 contains a pre-metered dose of a dry powder medication, which is to say that the medication is measured into the blister pocket 243 and the blister 240 sealed at manufacture, before delivery to the patient. In the present embodiment, the capacity of the blister pocket is about 120 microliters, and a dose of about 25 micrograms of medication, having a volume of about 30 microliters is held in the pocket.

The medication stored in the pocket comprises an aerosolizable, inhalable, dry powder blend of an inhaled corticosteroid (ICS), fluticasone propionate, and a long-acting bronchodilator, salmeterol xinafoate, blended with a lactose carrier. The blend is suitable for the treatment of asthma, and chronic obstructive pulmonary disease (COPD).

When the unit dose blister 240 is inserted into the seat 234, the annular collar rests against the upper surface of the platform 236 to align the unit dose blister 240 vertically within the device. The cylindrical recess 238 of the seat 234 has a diameter slightly larger than the concave blister pocket 243 where it meets the flat annular collar 246. This ensures that the recess 238 aligns the unit dose blister 240 horizontally to ensure it locates coaxial to the recess 238, as shown in cross-section at FIG. 8.

The cylindrical recess 238 is provided at its base with a concave recess 248 which provides a visual cue to the user of the device that the unit dose blister 240 should be inserted into the seat 234 for use.

Storage

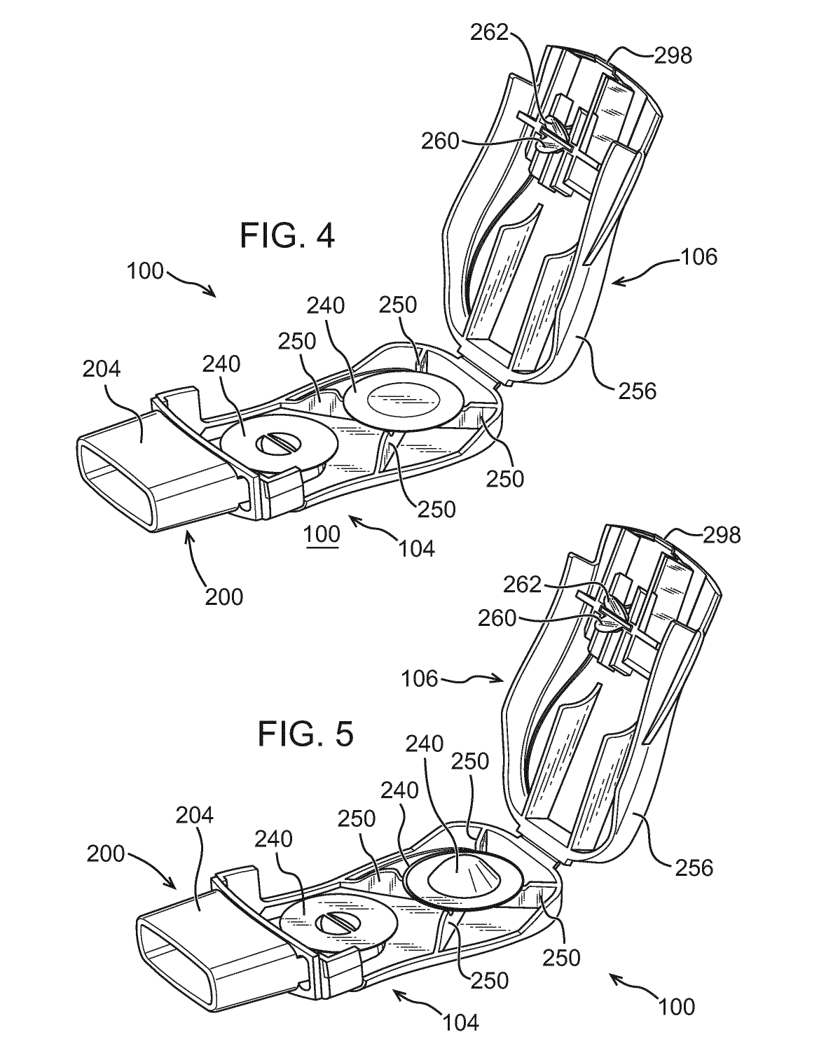

The bottom plate 224 of the housing 104 is further provided with a cruciform array of four equi-spaced reinforcing ribs 250 which project from the bottom plate 224 in the region next to the hinge 110. The ribs 250 increase the rigidity of the base 106 and are shaped on their upper surface a to define a central well 252 adapted to receive a second unit dose blister 240 for storage, as shown at FIG. 4, and a further third unit dose blister 240 stacked on top of the second unit dose blister in lid-to-lid arrangement, as shown at FIG. 5.

Structure—Lid

Referring back to FIG. 2, the housing lid 108 comprises a top plate 254 which has essentially the same plan form as the bottom plate 224 of the housing base 106. The top plate 254 is bowed to bulge outwards away from the cavity 111. The top plate 254 has an upstanding perimeter wall 256 which extends upwards from the periphery of the top plate, and surrounds three sides of the top plate 254. The upper surface of the lid perimeter wall 256 forms the abutment surface 114.

The base perimeter wall 226 and lid perimeter wall 256 are shaped to inter-engage when the housing 104 is in the ‘closed position’ to provide a substantially continuous wall of constant height between the bottom plate 224 and top plate 256 of the housing 110. The top plate perimeter wall 256 is omitted in the region which meets the mouthpiece bulkhead 228 so that the bulkhead 228 directly abuts the internal surface of the top plate 254.

Structure—Punch

Also shown at FIG. 2, the housing lid 108 is provided with a punch 258 which projects from the inner surface of the top plate 256 such that pivoting the lid 108 from the open position (e.g. FIG. 2) to the closed position (e.g. FIG. 1) drives the punch 258 through the lid 244 of the unit dose blister 240.

In use, as will be described in more detail subsequently, a patient inhales through the mouthpiece 102 to create an airflow through the device 100 so that air flows from the inlets 233 in the mouthpiece bulkhead 228, to the blister pocket 242, and onwards to the mouthpiece.

Henceforth, structures which lie on this airflow path will be described relative to the airflow. Generally, it will be understood that “downstream” features lie closer to the mouthpiece 200 than corresponding “upstream” features.

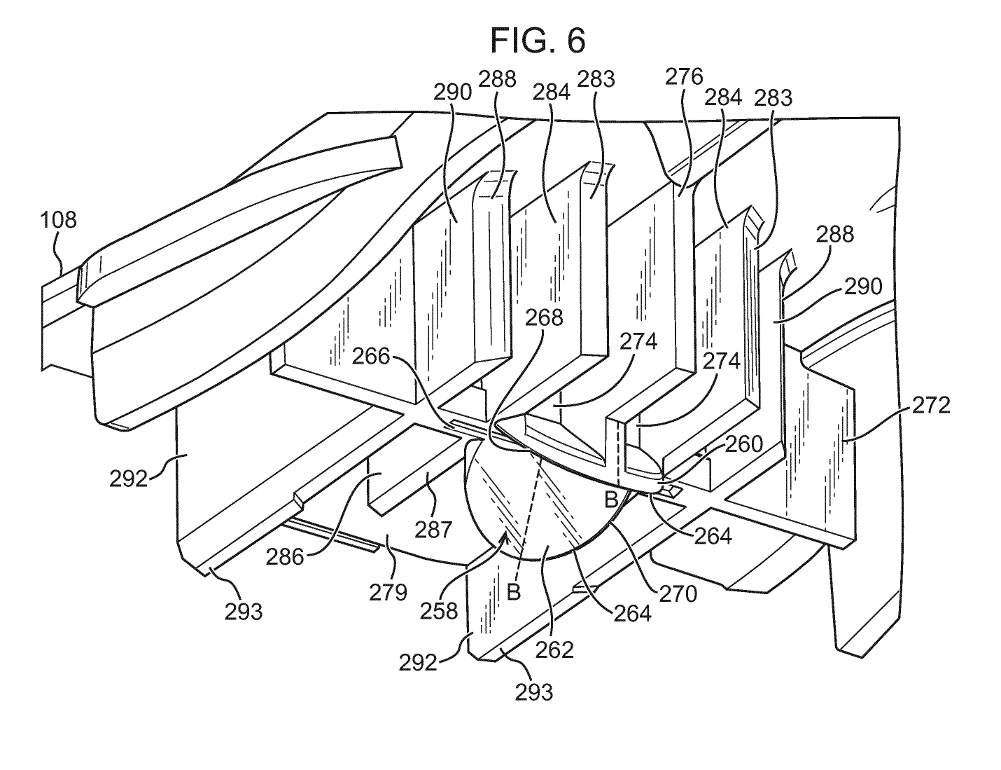

Turning to FIG. 6, part of the inner surface of the housing lid 106, and in particular the punch 258 is shown in more detail. The punch 258 comprises an upstream blade 260 and a downstream blade 262. Each blade comprises a curved, preferably semi-oval, planar element, having a curved, free, cutting edge 264 which extends from a first end 266 of a linear base 268 which is common to both blades 260, 262. The cutting edge 264 curves back on itself to return to a second end 270 of the linear base 268.

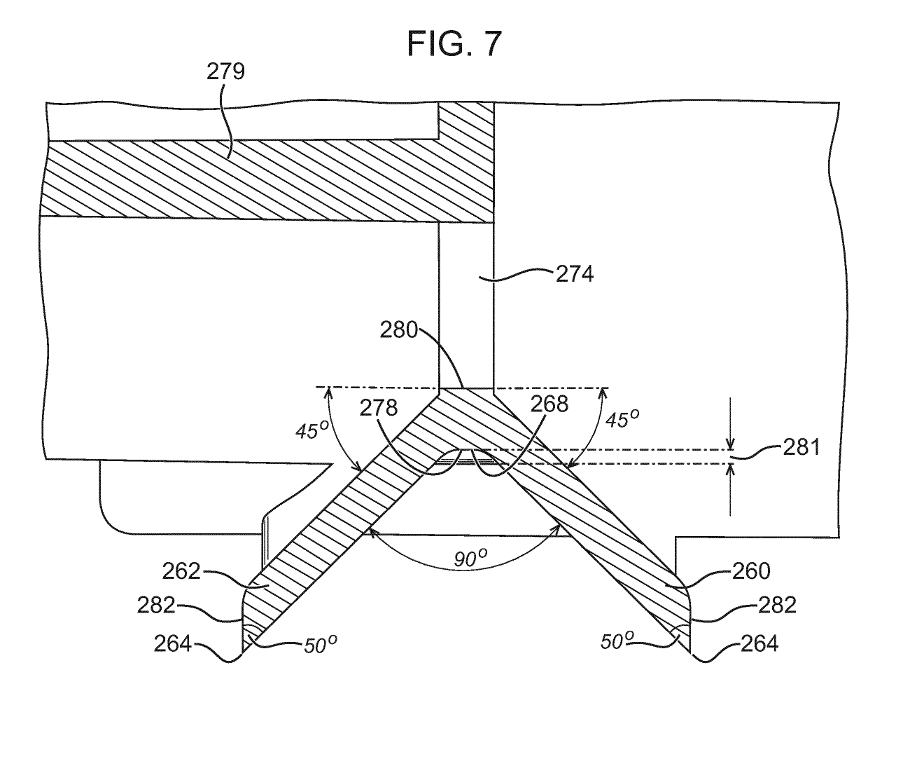

Referring now to FIG. 7, there is shown a schematic section view of the punch 258, on the dashed line B-B of FIG. 6. The blades 260,262 of the punch 258, are arranged in a back-to-back configuration and project from the common linear base 268 in opposite, diverging, directions. Each blade 260, 262 cantilevers from the inner surface of a punch bulkhead 272, discussed in more detail below, at an angle of about 45°, such that the included angle between the blades 260, 262 is about 90°. The blades 260, 262 are each oriented such that they traverse the longitudinal axis 116 of the device 100, and the common linear base 268 also traverses the longitudinal axis 116.

Turning back to FIG. 6, the linear base 268 of the punch 258 is provided by a punch bulkhead 272 which depends from the housing lid 108, into the cavity (not shown) and which traverses the top plate 254.

The bulkhead 272 is provided with an air inlet aperture 274 located above the punch 258. The aperture is shown in section at FIG. 7. The aperture 274 is bifurcated by a central buttress 276 which extends between the top plate 254 and the piercing blades 260, 262. The central buttress 276 is oriented parallel to the longitudinal axis 116 of the device 100. An upper boundary of the aperture is defined by a horizontal wall 279 which extends downstream from the aperture 274 such that it abuts the mouthpiece bulkhead 228 in the closed position of the device 100 as shown in cross-section at FIG. 8.

Turning back to FIG. 7, the blades 260, 262 of the punch 258 are arranged in an inverted ‘V’ formation. An internal apex 278 is defined by the common linear base 268 of the first and second blades 260, 262. A corresponding external apex 280 is defined at the external join of the first and second blades 260, 262, which forms a lower boundary of the air inlet aperture 274.

The common linear base 268 (internal apex 278) is set back into the punch bulkhead 272 by a predetermined distance 281 so that, in the closed position, it is spaced apart from the unit dose blister lid 244, which otherwise contacts the bulkhead 272.

The cutting edge 264 of each blade 260,262 is provided by a bevelled edge 282 applied to the end of each piercing blade 260,262. In the embodiment shown in FIG. 7, each blade 260,262 is of constant wall thickness, with a 50° bevel applied to the free end such that each cutting edge 264 has an included angle of about 50°, as shown at FIG. 7.

Structure—Buttresses

Turning back to FIG. 6, the side boundaries of the air inlet aperture 274 are provided by a first and second dosing channel buttress 283, which lie equi-spaced on either side of the central buttress 276. Each dosing channel buttress 283 extends in the direction of the longitudinal axis 116, parallel with the central buttress 276, and has an upstream section 284 and a downstream section 286. The upstream section 284 depends from the top plate 254, and the downstream section 286 depends from the horizontal wall 279. Together, the upstream section 284 and downstream section 286 define a wall having a flat lower surface 287 which abuts the blister lid 244 when the housing 104 is in the closed position.

A first and second outer buttress 288 is provided on either side of the central buttress 276, outboard of the dosing channel buttresses 283. Each outer buttress 288 has an upstream section 290 of substantially the same length as each upstream dosing channel buttress 284, and a downstream section 292 which is longer than the downstream inner buttress sections 286 and which abuts the mouthpiece bulkhead 228, when the inhaler device 100 is in the closed position (e.g. FIG. 1).

The outer buttresses 288 depend from the top plate 254 and project downwards to define a wall which has a substantially flat lower surface 293 over a region which abuts the unit dose blister 240.

Beyond this region, the outer buttresses 208 are extended downwards such that they abut the seat platform 236 directly.

Referring to FIG. 8, a dosing channel 412 is defined, upstream of the punch bulkhead 272, by the top plate 254, the upstream dosing channel buttresses 284 (only one shown), and blister lid 244. The dosing channel continues downstream of the punch bulkhead 272, and is defined downstream of the bulkhead 272 by the horizontal wall 279, downstream outer buttresses 292 and the blister lid 244 and raised platform 236.

In more detail, the upper wall of the dosing channel 412 is provided by the top plate 254, upstream of the punch bulkhead 228, and by the horizontal wall 279, downstream of the bulkhead 228. The sidewalls of the channel 412 are provided by the upstream dosing channel buttresses 284, upstream of the punch bulkhead 228, and by the downstream outer buttresses 292, downstream of the punch bulkhead 272. Finally, the lower wall of the dosing channel 412 is provided by blister lid upstream and downstream of the punch, and also by the raised platform 236, downstream of the bulkhead 228.

It will be appreciated that the dosing channel 412 is formed by a combination of features located upon the housing lid 108 and features located upon the housing base 106 and also by the unit dose blister 240, when a unit dose blister 240 is loaded in the device 100 and the device is configured in the closed position. In order to control airflow through the device 100 in use, it is important that entry of said airflow into the duct 412 should be controlled. Hence the abutment between the upstream dosing channel buttresses 284 and the unit dose blister lid 244 is important as it avoids the creation of a leak path for leakage of air into the duct 412. Similarly, the abutment of the downstream buttress section 292 against both the unit dose blister lid 244 and raised platform 236 (of the blister receiving seat 234) also avoids the creation of a leak path. Finally the abutment between the horizontal wall 279 and mouthpiece bulkhead 228 in the closed position of the device 100 as shown in cross-section at FIG. 8 also avoids the creation of a leak path.

By using the blister lid 244 to define part of the flow path through the device, the amount of material required to manufacture the device is reduced.

Referring back to FIG. 2, the lid top plate 254 has a proximal end 294 from which the hinge 110 depends, and a distal, opposite, end 296 provided with a projecting tab 298. The tab 298 cooperates with a recess 299 formed on the cavity-facing surface of the mouthpiece bulkhead 228 (shown in dashed outline at FIG. 3) to releasably lock the lid 108 in the closed position.

Use—Piercing

In use, a user moves the lid 108 from the closed position to the open position shown in FIG. 2 and inserts the unit dose blister 240 into the recess 238. The collar 246 of the unit dose blister 240 rests on the raised seat platform 236, and the blister pocket 243 centralizes within the seat 234 due to the geometry of the seat 234 as discussed previously. Preferably, the mouthpiece cover 102 is left attached to the housing 104 such that the mouthpiece 200 remains covered during the piercing process to prevent contamination of the mouthpiece 200 and to prevent the escape of medication from the mouthpiece 200.

The user then moves the lid 108 into the closed position by pivoting it about the hinge 110. As the lid 108 closes, it is brought into a first position in which the lid tab 298 abuts the mouthpiece bulkhead 228, which displaces the lid 108 towards the hinge 110. As the lid 108 is pivoted further towards the closed position, the upstream piercing blade 260 is brought into contact with the blister lid 244 such that the free cutting edge 264 of the upstream blade 260 engages and then pierces the puncturable disk region 247 of the lid 244. As the user continues to close the lid 108 against the housing base 106, the downstream piercing blade 262 engages and then pierces the puncturable disk region 247 at a location downstream of the first piercing. This sequential piercing of the blister lid 244 is intended to reduce the peak operating force required by the user when closing the lid 108 and thereby reduces the strength required by a patient to operate the device 100. This helps to facilitate operation of the device 100 by patients with reduced hand strength.

Finally, the lid 108 is brought to the closed position such that the upstanding perimeter wall 256 of the lid 108 bears against the upstanding perimeter wall 226 of the base 106. Finally, tab 298 is received by the slot 299 causing the lid 108 to move away from the hinge 110 towards mouthpiece 200 in the direction of the longitudinal axis 116, relative to the base 106.

This final longitudinal movement of the lid 108 relative to the housing base 106 causes the piercing blades 260,262 to further enlarge apertures formed in the lid by the blades 260, 262.

Because the blades only pierce the lid 244 of the unit dose blister 240, which is considerably thinner than the base sheet 242, the loads applied to the device 100 are reduced, enabling reduced cost, complexity, and weight.

Pierced Lid Geometry

Referring again to FIG. 8, there is shown an offset section through the inhaler device 100 on the section marked C-C in FIG. 1. The device 100 is loaded with a unit dose blister 240, which has been pierced by moving the housing lid 108 from the open position to the closed position such that the first and second cutting blades 260, 262 have pierced the lid 244 of the blister 240. In this closed position, the internal apex 268 of the punch is spaced above the lid foil 244, separated by a predetermined gap 300 of about 0.2 mm. The separation is created by the setback of common linear base 268 and ensures that, the punch creates a separate upstream inlet aperture 302 and downstream exit apertures 304 in the blister lid 244 by the following method;

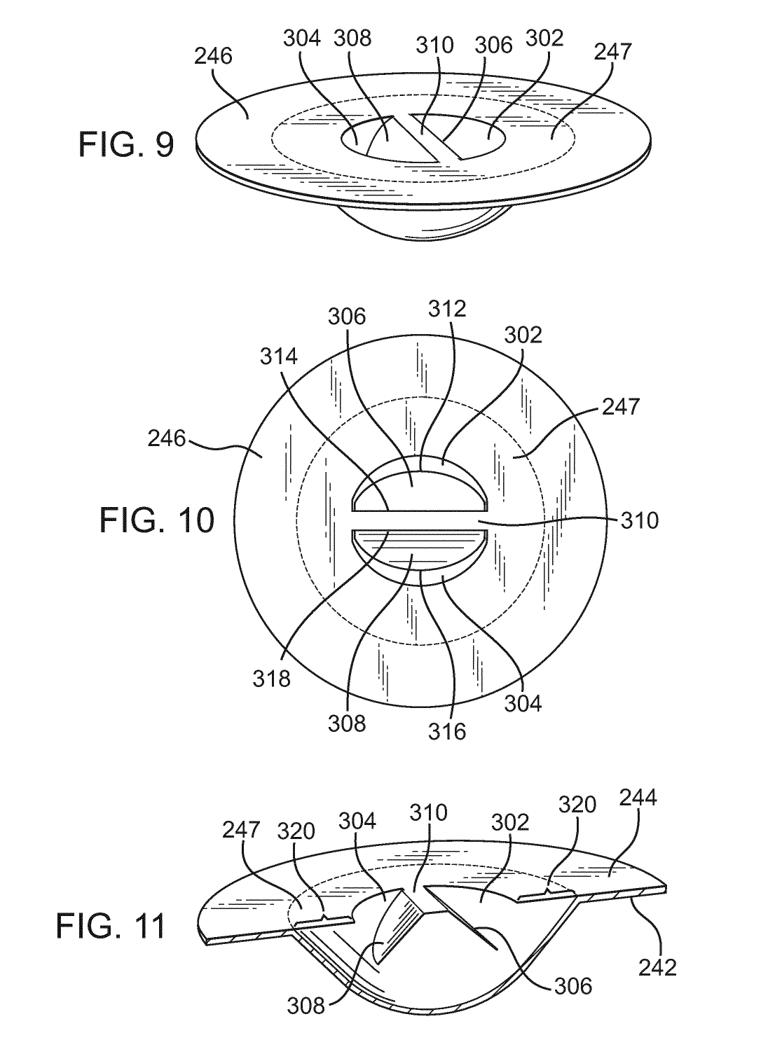

As the punch 258 engages the blister lid foil 244 during the piercing process, the upstream blade 260 cuts out an upstream flap 306 of lid material, shown at FIG. 9, which is displaced into the pocket 243 by the piercing blade 260. Similarly, the downstream blade 262 cuts out a downstream flap 308, shown at FIG. 9, which is displaced into the pocket 243. Both flaps 306, 308 tend to spring back to their original position, with the effect that they are biased against the lower surface of each blade 260, 262.

Because the internal apex 278 formed by the blades 260, 262 is spaced away from the lid 244 in the closed position of the device 100. The flaps 306, 308 are retained by, and depend from, a bridge 310 region of the lid 244. This can be seen more clearly with reference to FIG. 9 which shows the pierced unit blister dose 240 in isolation. As can be seen, the central bridge 310 is formed by lid material 244 which is left uncut between the flaps 306, 308, after the piercing process. The bridge 310 extends across the puncturable disk region 247 meeting the lid foil portion of the collar region 246 at diametrically opposing points.

Turning to FIG. 10, which shows a plan view of the blister lid 244 of FIG. 9, the upstream flap 306 has a curved free edge 312, and a linear fold region 314 which depends from the central bridge 310. The upstream flap 306 is deflected into the blister pocket 243 by bending at the fold region 314 to create the upstream aperture 302, in the lid foil 244.

Similarly the downstream flap 308 has a curved free edge 316, and a linear fold region 314 which depends from the central bridge 310. Again, the flap 308 is deflected into the blister pocket by bending at a fold region 318 to create the downstream aperture 304 in the blister lid 244.

Turning now to FIG. 11, there is shown a section of the unit dose blister 240 of FIG. 9, bisected along the longitudinal axis 116 of the device 100.

The width of bridge 310 is approximately 10% of the puncturable disk 247 i.e. the diameter of blister lid 244, excluding the annular collar 246. Each flap 306, 308 has a maximum length, normal to the linear fold region 314, of about one quarter of the diameter of the puncturable disk 247.

The geometry of the punch 258 is arranged so that the flaps 306, 308 and supporting bridge 310 are formed in a central region of the puncturable disk 247. The flaps 306, 308 do not extend to the edge of the disk 247 and this leaves an undisturbed annular overhang 320 of unsupported lid foil 244 projecting radially inwards from the wall of the blister pocket 243. This annular ‘overhang’ 320 projects inwards from the outer edge of the blister pocket 243, to a distance of about 20% of the diameter of the puncturable disk 247 i.e. the unsupported region of the lid foil 244. In the present example, a continuous annular overhang 320, is provided such that the total proportion of the puncturable disk 247 is about 40% of the diameter for any cross section of the pocket 240 excluding the bridge region, such that overhang comprises about 65% of the total area puncturable disk 247.

Relationship of Blister and Punch

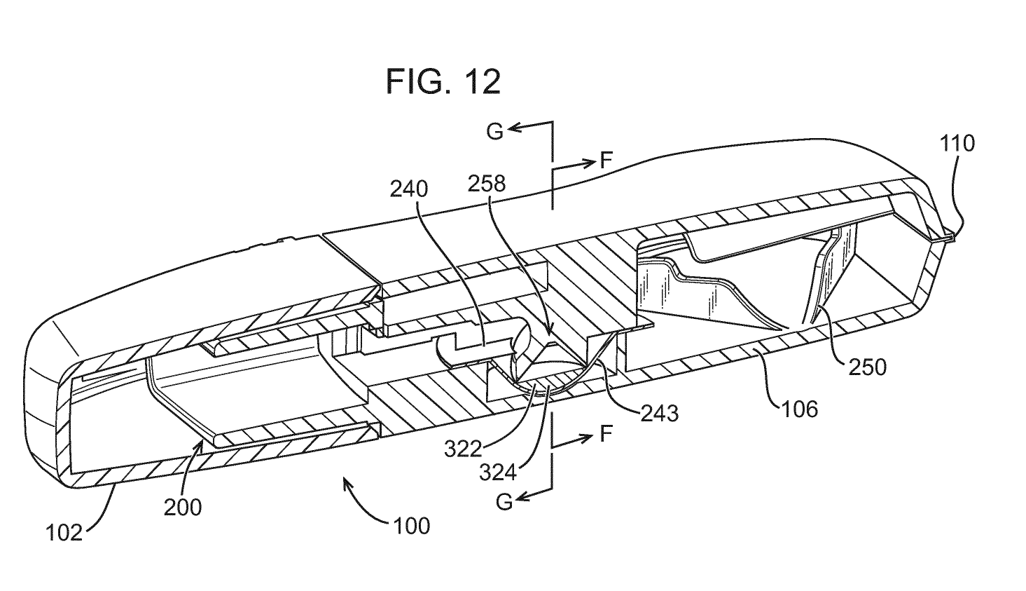

FIG. 12 shows a section through the device 100 along the longitudinal axis. The device 100 is shown configured in the closed position and loaded with a unit dose blister 240 which has been pierced by the punch 258. A medication 322 is located within a central region 324 of the pocket 243, bound in part by the upstream and downstream blades 260, 262. It will be understood that, because the pocket 243 is only about a quarter filled by volume with the medication 322, the majority of the medication 322 is held within this central region 324 after piercing of the blister 240.

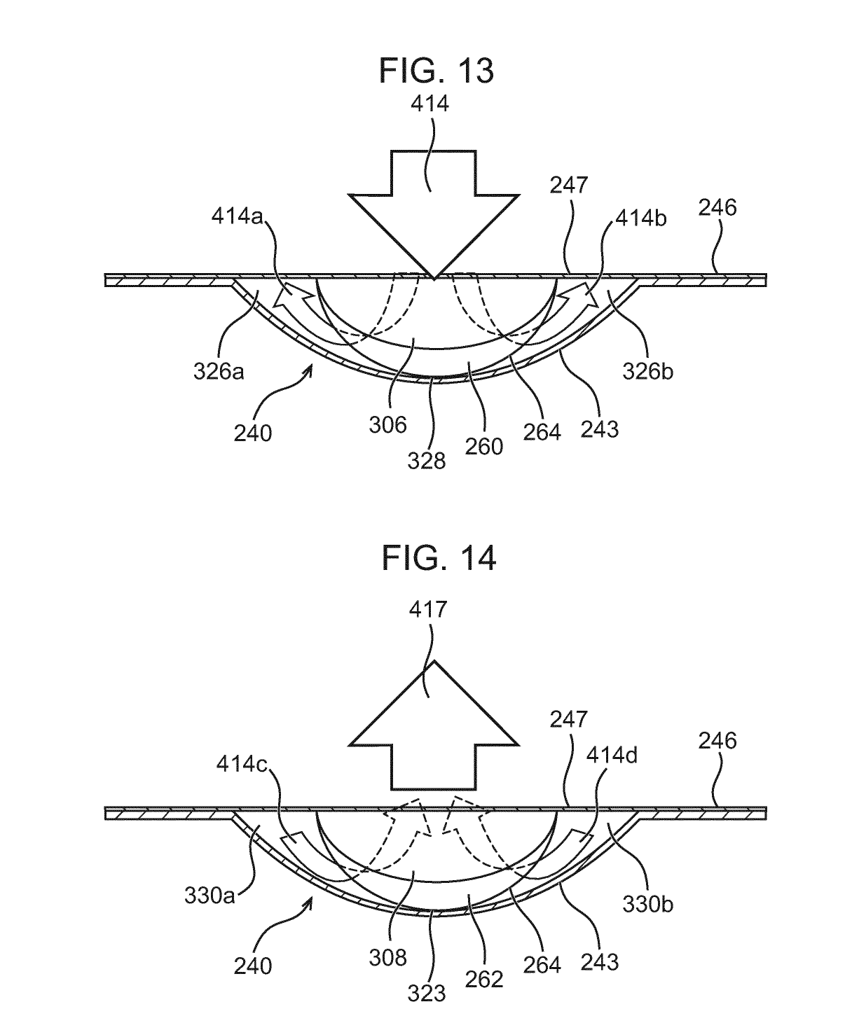

Turning now to FIG. 13 there is shown a view on the line F-F of FIG. 12 with the medication 322 omitted for clarity. This shows the upstream cutting blade 260 and upstream flap 306 as viewed from the central region 324 of the pocket 243, looking in the upstream direction i.e. away from the mouthpiece 200 in the direction of the longitudinal axis 116.

With the punch 258 inserted in the lid foil 244, the upstream blade 260 cooperates with the pocket 2434 to define an upstream channel 324a, 324b through which air can enter the central region 324 of the blister pocket 243. The upstream blade 260 contacts the blister pocket 243 at a point 328 approximately halfway along the cutting edge 264 of the blade 260 to divide the channel 326a, 326b in two. On each side of this contact point 328, the cutting edge 264 is gradually spaced apart from the wall of the blister pocket 243 to provide separate routes 326a, 326b for the air to enter central region 324 of the pocket 243.

Turning to FIG. 14, there is shown a view on the line G-G of FIG. 12 with the medication omitted for clarity. This shows the downstream cutting blade 262 and downstream flap 308, viewed from the central region 326 of the blister pocket 243 in the downstream direction i.e. towards the mouthpiece 200 in the direction of the longitudinal axis. As with the upstream blade 260, the downstream blade 262 cooperates with the upstream aperture 304 (shown at FIG. 8) formed in the lid 244 by the piercing process to define a downstream channel 328a, 328b through which air is able to exit the pocket 243. The cutting edge 264 of the downstream blade contacts the blister pocket 243 at a point 330 approximately halfway along the cutting edge 264 to divide this channel 330a, 330b in two. Either side of the contact point 332, the cutting edge 264 is gradually spaced apart from the wall of the blister pocket 243 to provide separate routes for the air to exit from the central region 326 of the pocket 243.

Use—Inhalation & Airflow

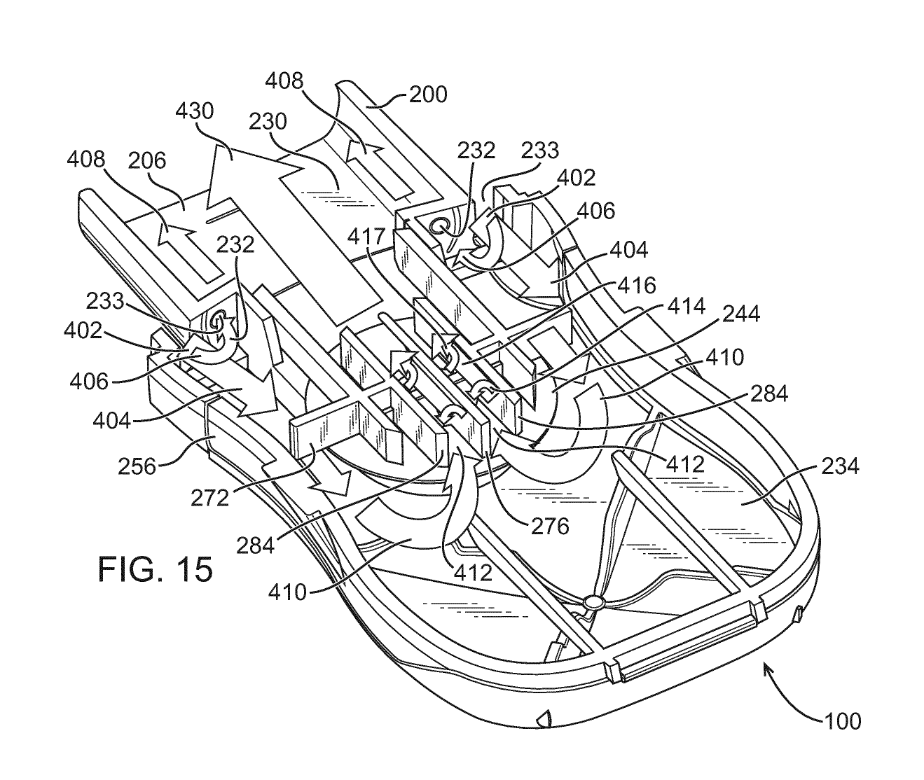

After the device 100 has been moved to the closed position shown in FIG. 8, the mouthpiece cover 102 is removed, and the user places his or her mouth over the mouthpiece 200. The patient then inhales via the mouthpiece 200 to generate an airflow through the device, as shown schematically by the arrows of FIG. 15.

FIG. 15 shows a perspective view, on the section indicated by line E-E at FIG. 8, through the lid of the device 100 in the closed position. The internal surface of the housing base plate 224 is visible, as well as the features of the housing lid 108 which define the dosing channel 412.

Inhalation by the user of the device 100 through the mouthpiece 200 creates a low pressure region within the mouthpiece 200 and, because the mouthpiece 200 is in flow communication with the inhaler cavity 111, a low pressure region in the cavity 111. As a consequence, air flows into the cavity 111 to create an air inlet airflow 402 at each air inlet 233 located in the mouthpiece bulkhead 228 on either side of the mouthpiece 200, external to the mouthpiece 200. Although it will be understood that some air will leak into the device 100 via the join between the housing base 106 and housing lid 108, a substantial majority of the air entering the device 100 does so via the air inlets 233.

Each inlet airflow 402 divides after entering the device 100 into a device-airflow 404 and a bleed-airflow 406. The device-airflow 404 continues into the device cavity 111 while the bleed-airflow 406 passes directly into the mouthpiece 200 via the first and second bleed holes 232. The bleed airflow 406 is intended to provide a ‘sheath’ 408 of clean air within the mouthpiece 200 to shield the internal surface 206 from powder laden air thus reducing deposition of powder on the inner surface of the mouthpiece.

Each device-airflow 404 passes into the device cavity 111, through the gap between housing base bottom plate 224 and the punch bulkhead 272, outside of the region that the bulkhead 272 abuts the unit dose blister lid 244. The device-airflow 404 then turns through 180° (410), and enters the dosing channel 412.

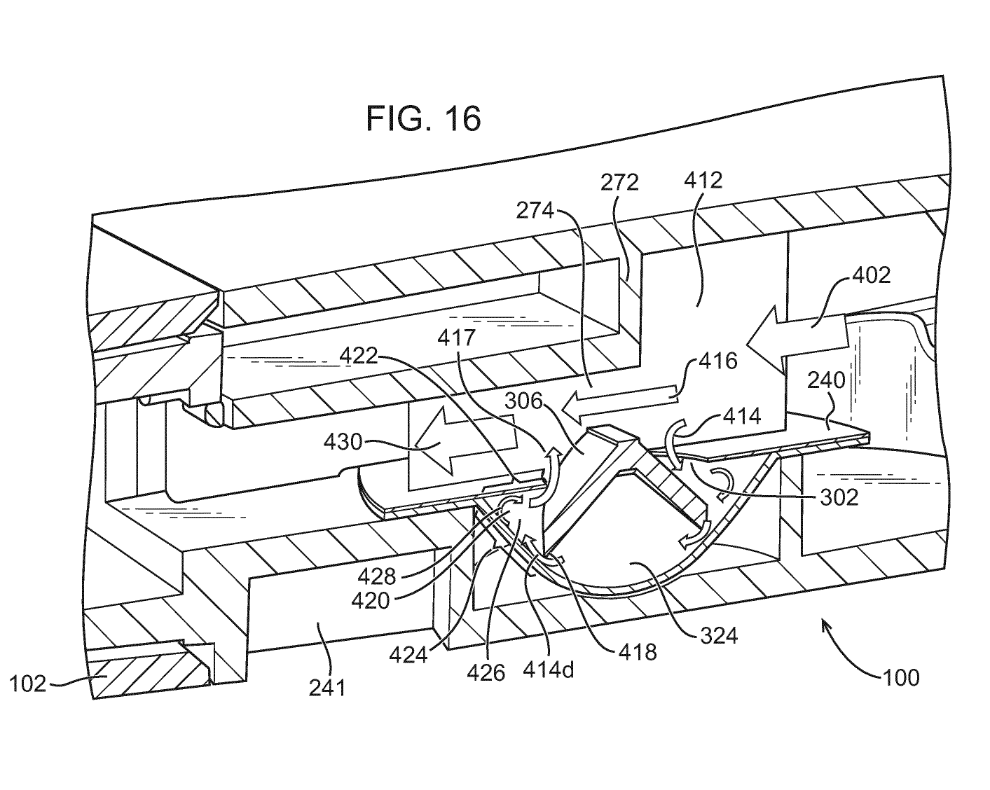

It will be appreciated from FIG. 15 that the dosing channel is bifurcated by the central buttress 276. Turning to FIG. 16, there is shown a section view through one of the two dosing channel halves created by this bifurcation. Airflow through the pierced unit dose blister 240 will now be explained with reference to FIGS. 14, 15 and 17 for clarity.

The device-airflow 402 enters the dosing channel 412, and is divided into a pocket airflow 414, which passes into and through the blister pocket 240, and a bypass airflow 416 which circumvents the blister pocket 240, via the inlet aperture 274 formed in the punch bulkhead 272. The bypass airflow 416 reduces the flow resistance of the device 100 by providing a greater total airflow through the device 100 than would be required solely for aerosolization of the medication (the pocket airflow 414). The reduced flow resistance ensures that the patient can inhale comfortably through the device 100, without undue restriction.

With reference to FIG. 13, the pocket airflow 414 enters the blister pocket 243 via the upstream inlet aperture 302 and then enters the central region 324 via the divided channel 326a, 326b. Two airflows 414a, 414b swirl around the upstream blade 260 and flap 306 creating a swirling airflow which aerosolizes the powdered medication (omitted for clarity) and tends to break up agglomerated particles of medication within the unit dose blister 240.

With reference to FIG. 14, the pocket airflow 414c, 414d, now containing aerosolized medication, exits the central region 324 via the divided downstream channel 330a, 330b. The pocket airflow 414c, 414d leaving the central region must speed up to maintain the mass flow through the divided downstream channel 330a, 330b.

Turning back to FIG. 16, the divided downstream channel 330a, 330b provides a nozzle 418 which directs and accelerates the airflow 414c, 414d leaving the central region into a trapping region 420 which is formed by the combination of the overhang 320 adjacent the downstream aperture 304, and the adjacent region of the blister pocket 243. The overhang 320 and adjacent region of the blister pocket 243 act as baffles 422, 424, which create a torturous path 426 that the exiting flow 414c, 414d must negotiate in order to exit the blister 240 via the exit aperture 304 and join the bypass flow 416.

Together, the nozzle 418 and trapping region 420 create a filter 428; whilst air is readily able to negotiate the torturous path 426, particles of medication aerosolized in the airflow 414c, 414d leaving the pocket 423 are more dense than air and, depending on their size, less able to follow the torturous path 426. In particular, it is observed from CFD analysis that the present geometry will retain a majority of 50 micron particles whilst allowing a majority of 5 micron particles to exit the pocket 243. This is beneficial as 50 micron particles do not travel well into the patient lung and tend to be deposited in the throat where they are ineffective, and can create an unpleasant taste for the patient. On the other hand, 5 micron particles are well sized for onward travel to the patient lung, resulting in effective delivery of medication to the lung.

The combination of pocket airflows 414c, 414d combine downstream of the central region 324 to form a powder laden pocket exit flow 417 which leaves the pocket 243 via the downstream aperture 304 it is directed into the bypass airflow 416 at an angle of approximately 90° thereto. This generates shear where the pocket exit airflow 417 and bypass airflow 416 meet which helps to further break up any undesirably large particles of medication that have escaped the blister pocket 243 As a consequence, the amount of medication delivered to the user in a useable form, e.g. 5 micron particles, is further improved.

The powder laden air 417 and the bypass airflow 416 recombine to form a device outlet flow 430 which passes through the slot shaped aperture 230 in the mouthpiece bulkhead 228 and on to the patient. Referring back to FIG. 15, the separation of the aperture 230 from the walls of the mouthpiece 200, and the bleed flow 408 is intended to prevent aerosolized medication held within the device outlet flow 430 from depositing on the internal surface 206 of the mouthpiece.

The apportionment of pocket airflow 414 and bypass airflow 416, as a percentage of the device-airflow 412, depends upon the size of upstream aperture 302 and downstream aperture 304 formed in the blister lid 244, and the size and shape of the inlet aperture 274 formed in the punch bulkhead 272. In particular, it is relatively straightforward to raise or lower the exterior apex 280 of the punch 258 to decrease or increase respectively the area of the inlet aperture 274 and correspondingly decrease, or increase the proportion of the device airflow 402 which bypasses the unit blister dose 240. The present device diverts about 15% of the device airflow 404 through the pocket 243 as pocket airflow 414, and the remaining airflow forms the bypass airflow 416.

Second Punch Geometry

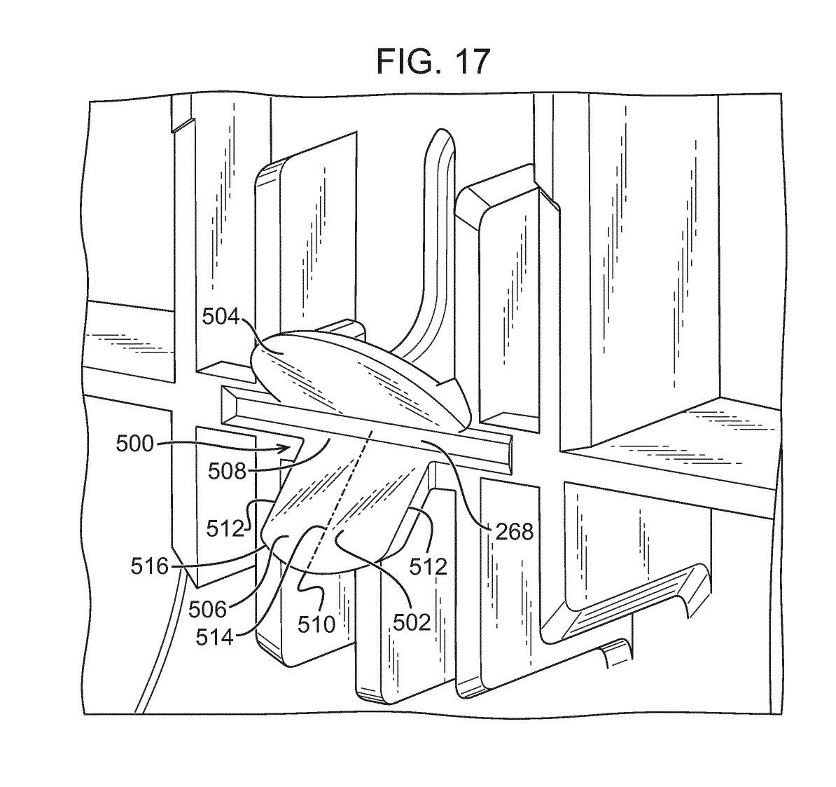

FIG. 17 shows a modified punch 500 for use with the device 100 in place of the punch 258 previously described. The structure of the inhaler device 100 is otherwise unaltered, hence like numbers will be used to identify like features.

The modified punch 500 comprises an upstream blade 502 and a downstream blade 504 which project from a common linear base 268 in opposite, diverging directions. The upstream blade 502 comprises an elongate tongue 506 having a proximal end 508 at the base 268 and a free distal end 510. The tongue 506 has straight parallel sides 512 equi-spaced either side of a central axis of projection 514. At the distal end 510 of the tongue, a convex curved cutting edge 516 extends between the sides 512 of the tongue 506.

The plan form of the modified upstream blade 502 can be said to comprises the blade 262 of the original punch 254, adapted by the removal of material outboard of parallel lines drawn equidistant from the centre line of the original blade 262. This results in an upstream blade 502 which is approximately 70% of the width of original upstream blade 260. The upstream blade 502 of the modified punch is 70% of the width of the downstream blade 500.

Applicant finds that this arrangement results in improved delivery of respirable medication in in vitro tests when compared with the geometry of the punch 258 already described. Surprisingly, it is observed that use of the modified punch 500 improves delivery over the original punch 258 and also provides improved delivery when compared with a third modified punch (not shown) in which the upstream blade and downstream blade are both narrowed as per the upstream blade 502 of the modified punch 500.

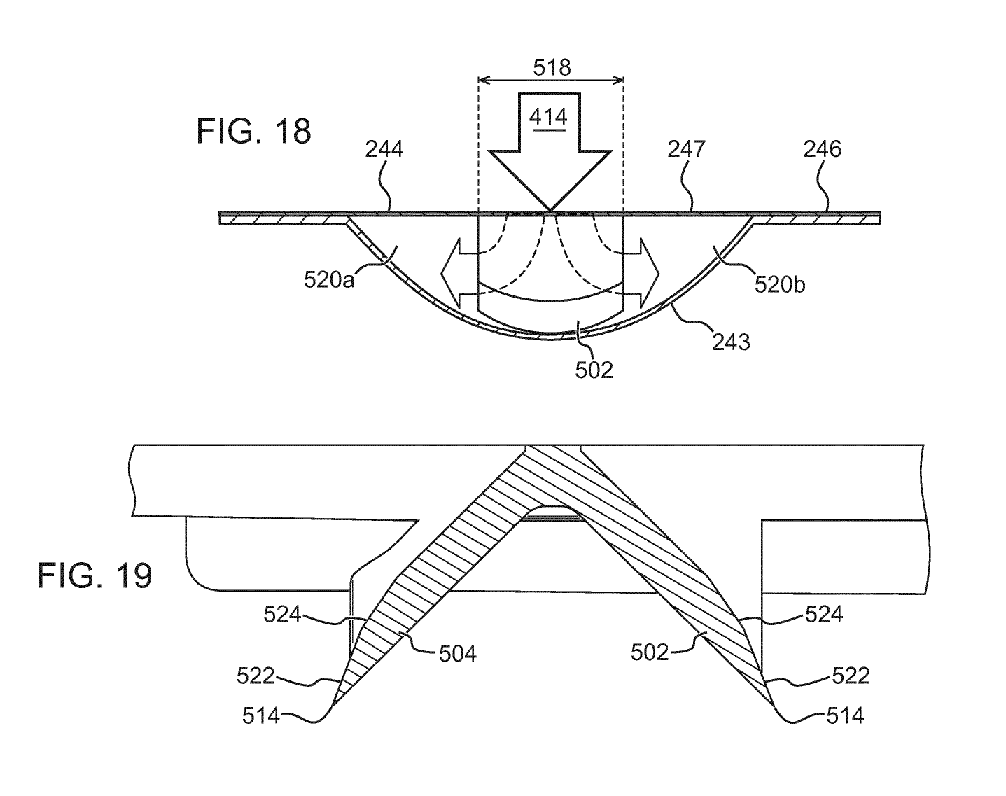

Turning to FIG. 18, there is shown a view from within the central region 247 of a unit dose blister 240, which has been pierced by the modified upstream blade 502 of the modified punch, in the upstream direction. In other words, FIG. 18 can be compared with FIG. 13 to see the relative difference created by the revised geometry of the modified upstream blade 502.

It is believed that the modified blade 502 gives an improvement in performance as the blade 502 creates an upstream aperture with smaller width, indicated by dashed lines 518, and hence reduced area. This increases the airflow velocity of pocket airflow 414 entering the pocket 243.

Also, the divided upstream aperture 520a, 520b created by the modified upstream blade 502 is larger than the upstream aperture 326a, 326b and this poses less of a restriction to airflow 414 into the pocket 243 which results in increased velocity of the airflow 414 entering the pocket for improved aerosolization of the medication held in the pocket 243. It is noted that the downstream blade 504 of the modified punch 500 is of the same geometry as the original punch 258 so that the modified punch 500 will still create the nozzle 418 and trap region 420 between the blade 504 and the pocket overhang 320.

Turning now to FIG. 19, the blades 502,504 of the modified punch 500 have a modified cross-section to improve airflow in to, and out of, the unit dose blister 240 during inhalation through the device 100.

The cutting edge 514 of each blade 502, 504 is provided by a bevelled edge 522 provided to the distal end of each blade 502, 504. The transition between each bevelled edge 522 and the constant thickness wall section of each blade 502, 504 is smoothed by the provision of a chamfer 524. The geometry provides a ‘twin-slope’ cross section to the surface of each blade 502, 504, which is exposed to pocket airflow 414, and powder-laden pocket exit airflow 417.

It will be understood by the skilled person that the ‘twin-slope’ cross-section can be applied to the punch 258, 500 of the device 100 independently of the revised geometry upstream piercing blade 504 described above. It will also be understood that the geometry of the ‘twin-slope’ cross-section can be applied to one, or both of the blades 260, 262, 502, 504 of the punch 258, 500.

Further Alternative Features

FIG. 20 shows an alternative mouthpiece 600 for use with the inhaler device 100 described hereinbefore. The mouthpiece 600 is a modification of the mouthpiece 200 described with reference to FIG. 3 and like reference numerals will be used to describe features common to both mouthpieces 200, 600.

The second mouthpiece 600 has an integrally formed mesh 602 which divides the slot shaped aperture 230 into a plurality of smaller holes 604 which promote turbulence to help break up agglomerated particles for improved delivery of medication to a user of the device 100.

A pair of parallel bars 606 extend vertically within the duct of the mouthpiece 600, between the upper and lower internal surface of the duct 204 adjacent the distal end 210 of the mouthpiece 600. The bars 606, and are equi-spaced from the centre line of the mouthpiece 600 such that the total separation 608 between the bars is less than the width of a unit dose blister 240. This prevents a patient misuse scenario wherein the patient attempts to insert the unit blister dose 240 into the device 100 via the mouthpiece 600.

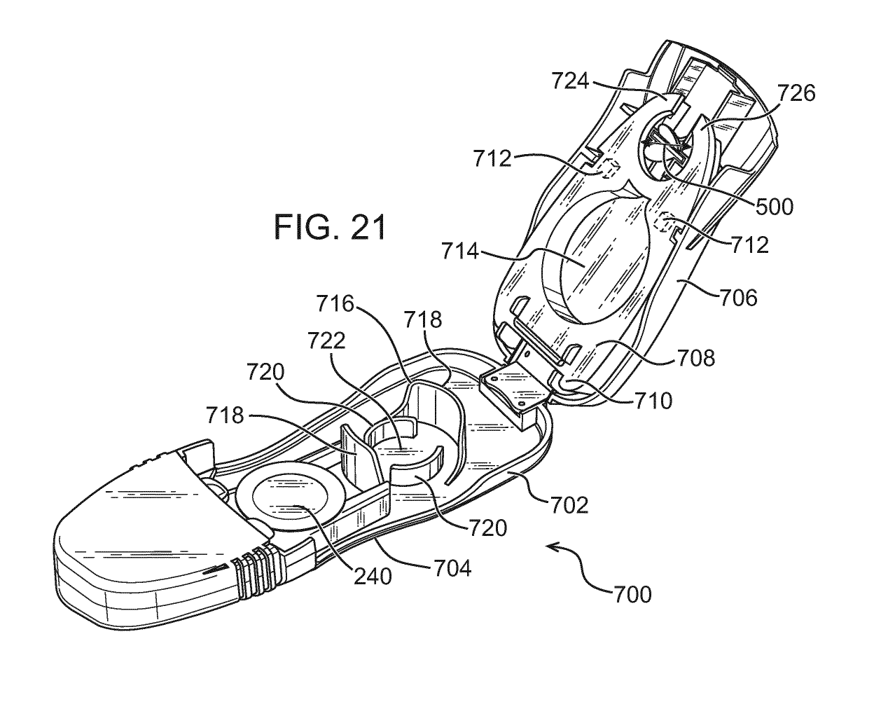

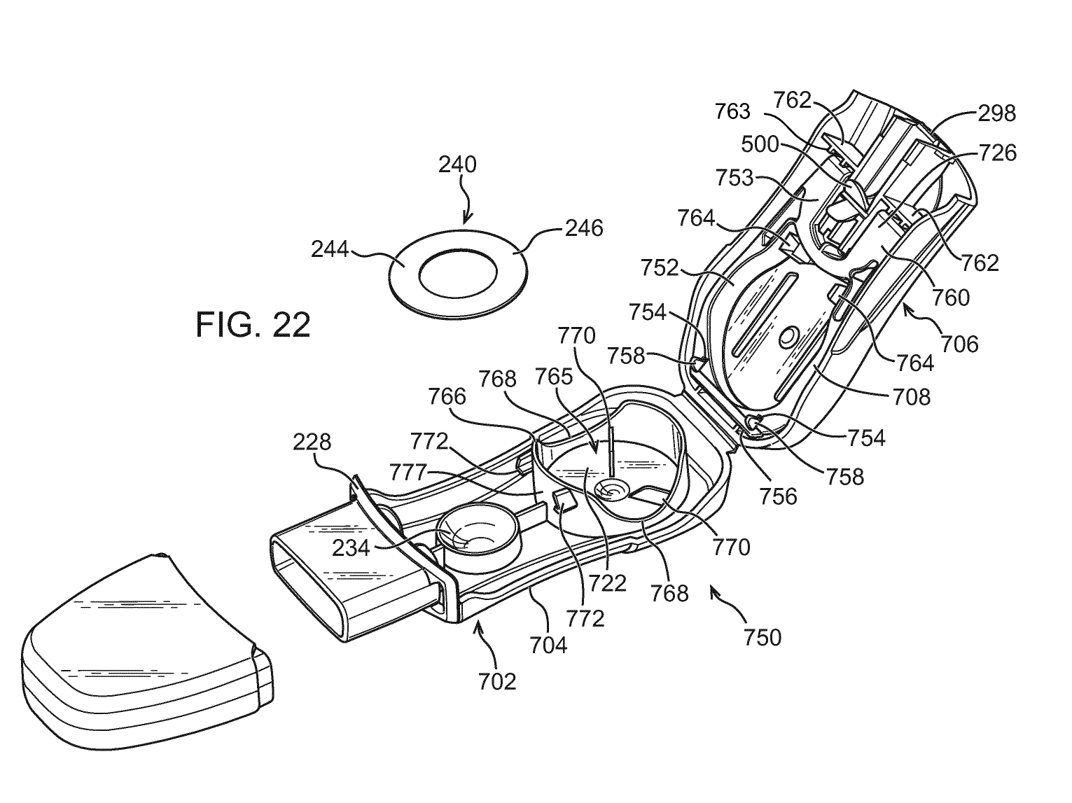

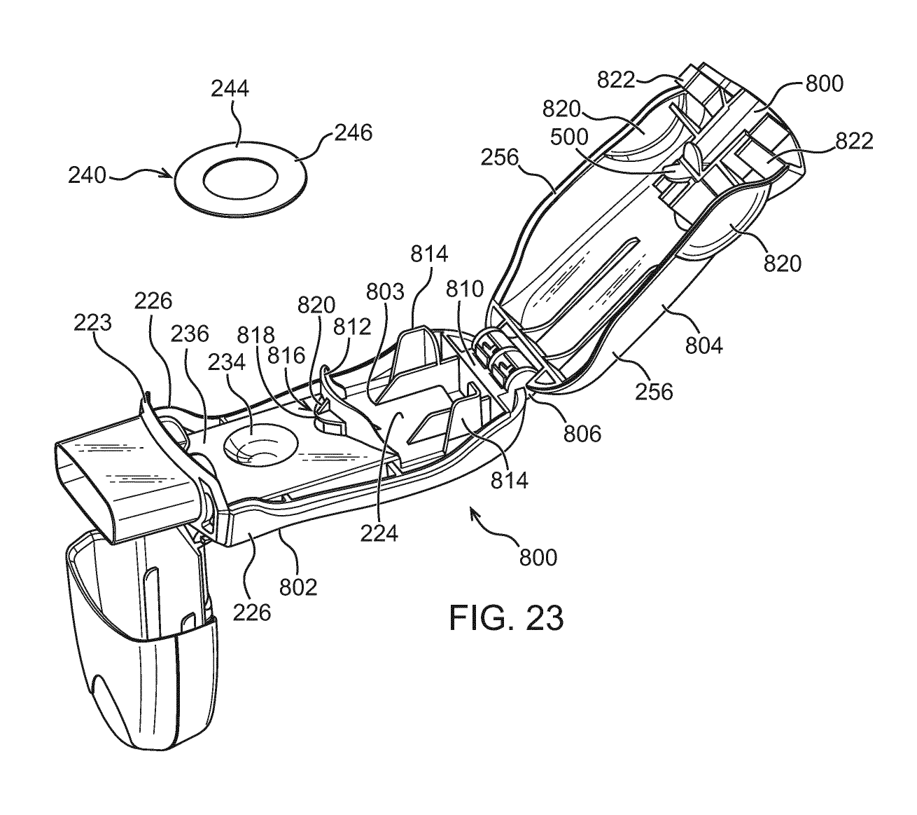

FIG. 21 shows an inhaler device 700 according to a further aspect of the present invention which is of essentially the same configuration as the inhaler device 100 shown in FIG. 1 et seq. The device 700 comprises a housing 702 having a base 704 and a housing lid 706 which are substantially equivalent to the housing base 106 and housing lid 108 of the device 100 shown FIG. 1 et seq. The housing lid 704 features a punch 500 as described hereinabove with reference to FIG. 17 and FIG. 18.

The housing lid 706 of the inhaler device 700 is provided with an ejector plate 708. This is provided to prevent a potential misuse scenario. The injector plate comprises a plate 708 which is pivotally mounted to the housing lid 704 via a pivot 710. The plate 708 is and sprung apart from, the housing lid 706 by a first and second plastic spring member 712 (shown in dashed outline). Optionally, the plate 708 may be formed as part of the single injection moulding used to form the housing 702 of the device 700, but in the embodiment shown, the plate 708 is formed separately. This provides the advantage of allowing the plate 708 to obscure the internal surface of the housing lid 706, thereby presenting an improved appearance to the user of the device 700. This is particularly beneficial where the housing 702 is produced by a ‘two shot’ injection moulding process in which two colours of plastic are moulded in the same tool, resulting in colour runs on the internal surface of a component.

The plate 708 is provided with a circular cut-out 714 of greater diameter than a unit dose blister 240 in order to accommodate a blister storage area 716 in the closed position of the device (not shown). The blister storage area 716 projects from the housing base 704 and is of a different configuration to the blister storage 250 shown in FIG. 4. The blister storage 716 comprises a first outer pair of opposing part-annular walls 718 and a second internal pair of opposing part annular walls 720, which define a central well 722 of equivalent function to the central well 252 shown in FIG. 2 and described previously. The circular cut-out 714 is of greater diameter than a unit dose blister 240, so that it does not interfere with storage of blisters in the central well 722 of the blister storage area 716.

The plate 706 is provided with a part annular collar 724 which partially surrounds the punch 500, which projects from the housing lid 704. The internal diameter 726 of this collar 724 is slightly larger than the diameter of the puncturable disc 247 so that, in the closed position of the device 700, it abuts against the collar 246 of the blister 240 and does not affect airflow into, and out of, the blister pocket 243.

In use, when the device 700 is moved to the closed position (not shown), with a blister 240 fitted, the ejector plate 708 is pushed against the housing lid 704, compressing both plastic springs 712. The punch 500 is thus able to pierce the lid foil 244 of the blister 240.

Upon opening the device 700, the springs 712 extend and the part annular collar 724 bears upon the blister 240 via the upper surface of the blister annular collar 246, pushing the blister 240 away from the punch 500. This ensures that the blister 240 separates from the punch 500 when device 100 is opened after piercing so that the used blister 240 is presented to the user sat in the receiving seat 234.

The ejector plate 708 avoids a potential misuse scenario in which, after piercing, the unit dose blister 240 can cling to the punch 258, so that, when the device 100 is opened after use, the blister 240 remains attached to the lid 108 via the punch 258. In this situation, it is possible that a patient fails to remove the used blister 240 from the punch and inserts a new blister into the seat 234. Upon closing the device, the old blister, still attached to the punch 258, can be forced through the lid foil 244 of the new blister.

Hence the patient could subsequently inhale through the old blister 240, without receiving any medication from the new blister. The pocket 243 of the old blister 240 will then prevent the powder from the new pocket being inhaled by the patient.

Finally, it will be understood that the unit dose blister can comprise more than one pocket 243 such that different medications can be separately stored for simultaneous delivery to the patient. For such a use, the device 100 will comprise a punch 258, 500 for each pocket 243.

With reference now to FIG. 22, an inhaler device 750 is shown which is a further development of the inhaler device 700 of FIG. 21. The device 750 is of broadly similar construction to the inhaler 700 of FIG. 21, and common features carry like reference numerals so that only differences between the devices 700, 750 are set forth with reference to FIG. 22.

The ejector plate 708 of the device 750 is provided with a central depression 752, instead of the circular cut-out 714 formed in the ejector plate 708 shown in FIG. 21. This central depression 752 performs the same role as the circular cut-out 714, which is to prevent the ejector plate 708 from fouling blisters stored within the housing 702, when the housing lid 706 is closed against the housing base 704. However, the depression 752 improves the strength of the ejector plate 708 by maximising the continuous region of material used within the available space, in particular within cavity defined by the housing 702 when closed.

The ejector plate 708 is further adapted to provide a U shaped collar 753 instead of the part annular collar 724 of the device 700 shown in FIG. 21.

The ejector plate 708 of FIG. 22 is pivotally mounted to the housing lid 706 via a pair of elongate slots 754 provided on either side of a first end 756 of the ejector plate 708. Each slot 754 receives a cooperating hook 758 which projects from the housing lid 706, and about which the plate 708 pivots.

A second, opposite end 760 of the ejector plate 708 slides against a buttress 762 provided to the housing lid 706. The buttresses 762 are each provide with hooks 763 which limit the pivoting movement of the plate 708 and prevents its detachment from the housing lid 706.

The ejector plate 708 is further provided with a pair of upstanding pawls 764 that project downwards, towards the housing base 704, when the lid 706 is moved to the closed position. The purpose of these pawls 764 will be explained in more detail below.

Turning now to the device housing base 706 of FIG. 22, the blister storage 765 of the device 750 comprises a single continuous annular wall 766 which is provided with a relief 768 on either side to allow a user to hold the sides of unit dose blisters (not shown) stacked within the central well 722 of the storage 765.

Three radial fins 770, of which only two are visible in FIG. 22, are provided at the base of the blister storage 765 to visually cue the user to the intended location for storage. The fins 770 are relieved at a central location to leave a storage well 722, which centralises a unit dose blister 240 within the storage 765 area.

The annular storage wall 766 is provided with a pair of latches 772 with project substantially parallel to the longitudinal axis of the device 750.

When the lid is 706 of the device 750 is closed, the projecting tab 298 of the lid 706 engage the slot 299 (not shown) in the mouthpiece bulkhead 228, to secure the lid 706 to the base 708. At the same time, the ejector plate pawls 764 engage the latches 772 protruding from the storage wall 766 to secure the ejector plate 708 to the base 704.

After use, the patient opens the device 750 to remove the spent blister by releasing the projecting tab 298 from the housing base 704. As the lid 706 is pivoted away from the base 704, the ejector plates 708 remains latched to the base 704 via the engagement of the pawls 764 with the latches 772. The pivoting attachment of the ejector plate 708 to the housing lid 706 allows the punch 500 to be withdrawn from the blister 240 with the lid 706, while the ejector plate 708 restrains the blister 240 in its seat 234. As the ejector plate meets the limit of its travel relative to the lid 706, further movement of the lid 706 away from the housing base 704 pulls the ejector plate 708 away from the base 704 causing the pawls 764 to disengage from the latches 772, allowing patient access to the blister 240. Hence the pawl 764 and latch 772 arrangement removes the need for the springs 712 of the device 700 of FIG. 21, simplifying the design.

FIG. 23 shows an inhaler device 800 according to a further aspect of the present invention. The device 800 is of broadly similar construction to the inhalers described previously and features are taken to be the same unless indicated otherwise.

The inhaler 800 comprises a separate housing base 802 and housing lid 804, joined by a snap-fit mechanical hinge 806. The mechanical hinge 806 is more complicated than a living hinge, and requires that the housing 802,804 is manufactured as two separate pieces. However patient-studies have found that the mechanical hinge provides a more useful visual cue for orientation of the device 800, than the living hinge used in other embodiments of the inhaler 800.

The device 800 comprises a unit dose blister storage area 808 defined between the hinged end 810 of the housing base 802 and a first curved bulkhead 812 which projects from the bottom plate 224. The bulkhead 812 is arcuate, curving towards the mouthpiece bulkhead 228. A pair of uprights 814, each of ‘L’ shaped cross section, project upwards from either side of the hinged end 810. An important aspect of the blister storage area 808 is that it is visibly differentiated from with the blister seat 234. In particular, the blister seat 236 provides a circular visual cue which mimics the shape of the unit dose blister form 240, thereby indicating where the blister 240 should be inserted for inhalation of a drug therefrom. In contrast, the blister storage area 808 provides a non-circular shape comprising a first curved surface 812 and at least two rectilinear forms 814. The clear visual differentiation between the storage area 808 and dosing area, seat 236 avoids a potential misuse scenario in which the patient inserts the unit dose blister dose 240 into the storage area 808 prior to closing the device, in the mistaken belief that the unit dose can be dispensed from this position.

Turning now to the platform 236, which provides the seat 234 for the unit dose blister 240, the platform 236 is enlarged when compared with the platform of the embodiment of FIG. 1 et seq.

In more detail, the platform 236 extends from the mouthpiece bulkhead 228 to the curved storage area bulkhead 812. The platform 236 is also widened to occupy a significant portion of the internal width of the housing base 802. The platforms 236 is sloped along its longitudinal edges so that a gap is formed between the annular collar 246 of the blister 240 towards and the platform 236 to facilitate removal of the blister 240 after use. The wide platform 236 provides a strong visual cue, along with the shape of the seat 234, that this region is the operative region for dosing from the unit dose blister 240.

In place of the ejector plate 708 used in the devices 700, 750 of FIGS. 21 and 22, the storage area bulkhead 812 is provided with a stripper hook 816 which projects towards the mouthpiece bulkhead 228, substantially parallel to the platform 236. The hook 816 is provided with a bevelled leading edge 818 which leads outwards towards the platform 236. A central buttress 820 extends between the upper surface of the hook 816 and the storage bulkhead 812 to reinforce the midpoint of the hook 814. The function of this hook 816 will be described below.

Each upstanding perimeter wall 256 of the housing lid 804 is provided with an outward facing, finger sized, indentation 820 on either side of the housing lid 804, approximately in line with the punch 500. The lid tab 298 of previous embodiments is omitted and a pair of legs 822 project from the inner surface of the housing lid 804. The legs 822 engage cooperating apertures (not visible) in the platform 236 when the lid 804 is closed against the base 802.

In use, the stripper hook 816 provides the same function as the ejector plate 708 of the devices 700, 750 shown in FIGS. 21 and 22 via a different route of action. Moreover, the hook 816 provides a simpler method of retaining the unit dose blister 240 within the housing seat 234 until the punch 500 has been withdrawn from the blister 240 via movement of the housing lid 804 away from the housing base 802.

In use, a patient inserts the blister 240 into the seat 234 at an insertion angle relative to the platform 236 such that the annular collar 246 of the tilted blister 240 slips into the gap defined between the platform 236 and the lower surface of the hook 816. As the blister 240 is pushed towards the annular storage wall 812, the concave blister pocket 243 (not shown) aligns with, and falls into, the blister seat 234 so that the outer rim 246 of the blister rests against the platform 236

The patient then closes the housing lid 804 against the housing base 802, causing the punch 500 to pierce the only the blister lid 244 as described previously. The patient can then administer the drug contained within the blister 240 by inhalation through the device 800.

To remove the empty blister 240 after use the patient opens the device 800 by gripping the indentations 820 of the lid 804. This causes the legs 822 to deflect towards the longitudinal axis of the device 800 causing them to unlatch form the housing base 802. This permits the lid 804 to be pivoted away from the housing 802.

Initially, the blister 240 moves with the lid 804 as described previously, because the blister 240 grips the punch 500 due to deformation of the blister lid 244 about the punch 500.

However, as the punch 500 moves along the arc prescribed by the hinge 806, its motion, and subsequent motion of the blister 240 is limited to upwards movement against the base of the stripper hook 816. Hence, once the blister 240 has contacted the lower surface of the hook 816, interaction of the blister 240 and hook 416, causes any further upward, opening, motion of the lid 804 to prise the punch 500 from the blister lid 244.

After the punch 500 has been fully withdrawn from the blister 240, the blister can be readily withdrawn from the device by reversing the insertion procedure described above i.e. tipping the blister 240 up from the platform 236 and then withdrawing it at the “insertion angle” to the platform 236.

In other words, the stripper hook 816 is configured to enable insertion and removal of the blister 240 in an insertion direction, which insertion angle is oriented at a substantially different angle to the angle of action of the punch 500 as it enters, and is withdrawn from, the blister lid 244. This ensures that the punch 500 cannot remove the blister 240 from the seat 234, but that a patient can readily do so, with minimal effort. In the present example, the insertion direction is approximately normal to the angle of action of the punch 500, wherein the angle of action of the punch is approximated to a straight line at the point of contact between the punch 500 and the blister 240.

Typically the insertion angle is between 5° to 20° to the platform 236. The stripper hook 816 of the device 800 of the present embodiment is arranged to define an insertion angle of 13°.

What is claimed is:

1. An inhaler device comprising;
a housing,
a mouthpiece,
a seat for receiving a unit dose blister, the unit dose blister comprising a blister pocket and a blister lid,
and a punch for piercing a lid of the unit dose blister received in the seat,
wherein the housing comprises a housing base and a housing lid pivotally joined by a hinge, such that the housing lid is pivotable from a first ‘closed’ position in which it abuts the housing base to define a cavity, to a second ‘open’ position in which the cavity can be accessed,
wherein the seat and the punch are adapted to lie within the cavity when the lid is in the first ‘closed’ position and
wherein moving the housing lid from the ‘open’ position to the ‘closed’ position causes the punch to pierce a lid of a unit dose blister received in the seat.

2. An inhaler device as claimed in claim 1 wherein the punch comprises a first piercing blade and a second piercing blade, and the first and second piercing blades are arranged such that movement of the housing lid from the second position to the first position causes the first piercing blade to engage and pierce the lid of the unit dose blister inserted in the seat before the second piercing blade engages and pierces the lid of the unit dose blister.

3. An inhaler device as claimed in claim 1 wherein the housing cooperates with the lid of the unit dose blister to form a dosing channel which divides an airflow through the device into a pocket airflow, and a bypass airflow, wherein the pocket airflow aerosolizes a powder held within the unit dose blister and the bypass airflow circumvents the unit dose blister.

4. An inhaler device as claimed in claim 1 further comprising a mouthpiece cover which can be attached to the inhaler device to enclose the mouthpiece, wherein the housing lid can be moved from the ‘open’ position to the ‘closed’ position without removing the mouthpiece cover such that the device cavity is substantially sealed from the external environment when the unit dose blister is fully opened by the punch.

5. An inhaler device as claimed in claim 4 wherein an inlet provided to the housing is covered by the mouthpiece when it is attached to the housing to enclose the mouthpiece.

6. An inhaler device as claimed in claim 4 wherein the mouthpiece depends from a first region of a curved mouthpiece bulkhead, and an air inlet is provided to a second region of the curved mouthpiece bulkhead, wherein the air inlet is set back from the first region of the bulkhead towards the inhaler cavity.

7. An inhaler device as claimed in claim 1 wherein the mouthpiece comprises a duct having a proximal end in flow communication with the housing, and a distal free end, wherein the housing communicates with the duct via an aperture which is smaller than the duct to minimise contact of powder laden air with an inner wall of the duct.

8. An inhaler device as claimed in claim 1 wherein the device further comprises a unit dose blister retainer which holds the blister in a predetermined relationship with the housing base as the punch is withdrawn from the blister by moving the housing lid from the ‘closed’ position to the ‘open’ position.

9. An inhaler device as claimed in claim 8 wherein the retainer comprises a hook.

10. An inhaler device as claimed in claim 9 wherein the hook is formed integral with the device housing.

11. A punch for an inhaler device, the punch adapted to pierce the lid of a blister comprising a base sheet defining a pocket, a pocket wall, and a lid covering the pocket,
wherein the punch comprises a downstream piercing blade adapted to pierce and define an exit aperture in the lid, wherein the exit aperture is spaced apart from the pocket wall to define an overhang region of the lid,
and wherein the downstream blade is further adapted to enter the pocket of the blister after piercing the lid to define a nozzle in cooperation with the wall of the blister pocket, such that when an airflow is generated through the pocket towards the exit aperture, the nozzle directs the airflow towards the overhang region of the lid so that it follows a torturous path before reaching the aperture.

12. A punch as claimed in claim 11 further comprising an upstream blade adapted to pierce and define an inlet aperture in the lid.

13. A punch as claimed in claim 12 wherein the downstream blade is wider than the upstream blade, preferably about 40% wider.

14. A punch as claimed in claim 12 or claim 13 wherein at least one piercing blade comprise a semi-oval planar element.

15. A punch as claimed in claim 12, wherein the first piercing blade and second piercing blades diverge from one another.
